(19) United States
(12) Patent Application Publication
(10) Pub. No.: US 2026/0346040 A1
(43) Pub. Date: Nov. 5, 2026

(54) X-RAY LINE SOURCE IMPLEMENTING CONFORMAL COLLIMATION, AND ASSOCIATED METHOD

(71) Applicant: NCX Corporation, Raleigh, NC (US)

(72) Inventors: Jian ZHANG, Durham, NC (US); Derrek SPRONK, Chapel Hill, NC (US)

(21) Appl. No.: 19/486,721

(22) PCT Filed: Jan. 22, 2025
(86) PCT No.: PCT/IB2025/050672

§ 371 Date: Nov. 21, 2025

Related U.S. Application Data
(60) Provisional application No. 63/623,669, filed on Jan. 22, 2024.

(51) Int. Cl. A61N 5/00; H01J 35/06; H01J 35/14; H01J 35/24

(52) U.S. Cl. CPC A61N 5/00; H01J 35/065; H01J 35/153; H01J 35/24

(57) ABSTRACT

An X-ray system for providing an X-ray dosage, includes an X-ray source including an anode and a field emission cathode device in spaced-apart relation with the anode and arranged to emit electrons toward the anode so as to form an elongate focal area on and extending along the anode and to actuate emission of X-ray beams from the elongate focal area directed toward a treatment field. A collimator device is disposed between the anode and the treatment field, wherein the collimator device is configured to shape the X-ray beams from the elongate focal area of the anode by filtering the X-ray beams to minimize a penumbra of the X-ray beams directed through the collimator device to interact with the treatment field. An associated method is also provided.

### BACKGROUND

### Field of the Disclosure

Aspects of the present disclosure are directed to X-ray imaging and therapy and, more particularly, to a system and method for X-ray imaging and targeted X-ray therapy implementing an X-ray line source with conformal collimation.

### Description of Related Art

An X-ray generating device (e.g., an X-ray tube) typically comprises a metal target at high potential on the order of tens of kV to hundreds of kV, an electron gun for providing a high intensity electron beam, and a vacuum envelope evacuated to micro-Torr or lower pressure levels. The electron beam from the electron gun is accelerated by the electrostatic field formed by the high potential on the metal target, and bombards the target, producing characteristic and Bremsstrahlung radiation (e.g., X-ray beams). The target can be made of different materials, geometries, and/or other features such that application-specific X-ray beams can be produced. Many existing X-ray tubes operate on this basic principle.

A major limitation in terms of the total power (and subsequently X-ray flux) available from a given X-ray tube assembly is the melting or thermal limit of the metal target (anode). Efforts directed toward X-ray tube target (anode) thermal management include, for example, forming the anode and supporting structures from an alloy with high thermal conductivity for rapid heat transfer (e.g., heat dissipation or cooling of the anode by heat conduction) as shown in FIG. 1A. The anode can also be made to rotate, reciprocate, or otherwise move cyclically to spread the electron beam power over larger area of the target (see, e.g., FIG. 1B). Another solution can involve, for example, forming a hollow channel in the anode through which a cooling liquid (e.g., water or oil) can be circulated with an associated heat exchanger to cool the liquid (FIG. 1C). Such methods can be sufficient to manage the heat load on the target (anode) in high power (kW) applications of the X-ray tube, but can add significant cost and footprint to the X-ray generating device.

Many existing X-ray sources (e.g., X-ray tubes) have been developed for imaging applications. For such imaging applications, the electron beam must be focused on a relatively small area on the anode, commonly referred to as the focal spot (see, e.g., FIG. 2A with the electron beam directed perpendicularly to the anode to generate a circular focal spot). While some of the energy imparted by the electron beam to the anode is converted to or generates X-rays, roughly 99% of that energy is converted into heat. The temperature of the focal spot on the anode thus rises, for example, at a rate proportional to the electron beam power divided by the focal spot area (otherwise referred to herein as “the power density”). This relatively small area/size of the focal spot, typically on the order of, for example, 1-10 mm² for imaging purposes (i.e., a relatively smaller focal spot size for imaging provides a higher pixel count proportional to the desired resolution of the X-ray image), prevents the electron beam power from being increased due to high power density and rapid temperature rise at the anode. As such, for X-ray applications that do not require small focal spots, the power density (and resulting temperature increase) can be decreased by establishing a relatively larger area of the focal spot, for example, on the order of 100-1000 mm². The size of the focal spot can be increased using a Line Focus X-ray source (LFX), for example, wherein the electron beam is arranged to obliquely interact with the anode such that the focal spot is extended in one dimension to create and approximate a focal line (see, e.g., FIG. 2B). However, if the same power density is to be achieved (i.e., remain constant), the increase in the area of the focal spot by two orders of magnitude requires a proportional increase in the total electron beam power.

Though the LFX concept may be a promising X-ray source alternative design for X-ray applications that require a relatively high X-ray output, such as phase contrast imaging (PCI) and/or radiation therapy (RT), one major issue for PCI applications is that there is very little spatial coherence in the dimension along the focal line. In the case of RT, radiation treatments have strict requirements on the acceptable level of X-ray dose/dosage that can be administered to otherwise healthy tissue. These issues often necessitate some X-ray sources to be accompanied by an adjustable collimator to shape the emitted X-ray beam to a well-defined field of view (FOV) or treatment field (see, e.g., FIG. 3A). However, when the focal spot dimension is as large or larger than the collimator opening or aperture (i.e., when using LFX), the penumbra (shown as a penumbra region in FIGS. 3A and 3B, but otherwise referred to herein as a penumbra, a penumbra region or a penumbra effect) extends the emitted X-ray beam beyond the FOV or treatment field/zone (see, e.g., FIG. 3B), thus posing a high risk of radiation-related side effects. In some instances, this issue could be addressed, for example, by establishing the focal spot/anode farther away from the collimator opening/aperture, but such as solution would reduce the X-ray flux or dosage delivered to the FOV/treatment field due to the inverse-square relationship of the X-ray flux/dosage with the distance from the FOV/treatment field. In other instances, the size of the collimator opening could be decreased. However, such a solution, though reducing the penumbra or penumbra region, would also reduce the overall efficiency of the X-ray treatment by blocking useful X-ray radiation from reaching the FOV/treatment field. Thus, a trade-off exists between the maximum X-ray flux available from an optimally collimated RT system and the minimum focal spot area of the X-ray source as to avoid melting or thermal degradation of the anode thereof.

Thus, there exists a need for an X-ray source capable of achieving a relatively high X-ray flux (e.g., a relatively high radiation dosage rate) while avoiding melting or thermal degradation of the anode thereof. It may also be desirable for such an X-ray source to be capable of operating in an imaging mode in which a small focal spot on the anode can be used to generate high resolution images, as well as to be capable of operating in a treatment mode in which a large focal spot/line on the anode can be used to achieve high radiation dosage rates desired for therapeutic cancer treatment.

### BRIEF SUMMARY OF THE DISCLOSURE

The above and other needs are met by the present disclosure which, in one aspect, provides a system for providing an X-ray dosage, the X-ray system comprising an X-ray source including an anode and a field emission cathode device in spaced-apart relation with the anode and arranged to emit electrons toward the anode so as to form an elongate focal area on and extending along the anode and to actuate emission of X-ray beams from the elongate focal area directed toward a treatment field; and a collimator device disposed between the anode and the treatment field, with the collimator device being configured to shape the X-ray beams from the elongate focal area of the anode by filtering the X-ray beams to minimize a penumbra of the X-ray beams directed through the collimator device to interact with the treatment field.

Another aspect of the present disclosure provides a method for providing an X-ray dosage, the method comprising forming an elongate focal area on and extending along an anode by emitting, using an X-ray source including the anode and a field emission cathode device in spaced-apart relation with the anode, electrons from the field emission cathode device toward the anode, the emitted electrons interacting with the anode to form the elongate focal area and actuating emission of X-ray beams from the elongate focal area toward a treatment field; and shaping, using a collimator device disposed between the anode and the treatment field, the X-ray beams from the elongate focal area of the anode by filtering the X-ray beams to minimize a penumbra of the X-ray beams directed through the collimator device to interact with the treatment field.

The present disclosure thus includes, without limitation, the following example embodiments:

Example Embodiment 1: A system for providing an X-ray dosage, the X-ray system comprising an X-ray source including an anode and a field emission cathode device in spaced-apart relation with the anode and arranged to emit electrons toward the anode so as to form an elongate focal area on and extending along the anode and to actuate emission of X-ray beams from the elongate focal area directed toward a treatment field; and a collimator device disposed between the anode and the treatment field, with the collimator device being configured to shape the X-ray beams from the elongate focal area of the anode by filtering the X-ray beams to minimize a penumbra of the X-ray beams directed through the collimator device to interact with the treatment field.

Example Embodiment 2: The system of any preceding example embodiment, or combinations thereof, wherein the elongate focal area on and extending along the anode defines a major dimension and a minor dimension, with the major dimension being greater than the minor dimension.

Example Embodiment 3: The system of any preceding example embodiment, or combinations thereof, wherein the field emission cathode device comprises one or more selectively-actuatable field emission cathode elements arranged to emit electrons toward the anode to form the elongate focal area on and extending along the anode.

Example Embodiment 4: The system of any preceding example embodiment, or combinations thereof, wherein the field emission cathode device comprises one or more selectively-actuatable field emission cathode elements arranged to each emit electrons toward different serially-contiguous focal spots or different serially-adjacent focal spots on the anode, the serially-contiguous or serially-adjacent focal spots forming the elongate focal area on and extending along the anode.

Example Embodiment 5: The system of any preceding example embodiment, or combinations thereof, wherein the field emission cathode device comprises one or more selectively-actuatable field emission cathode elements arranged to emit electrons toward different focal spots on the anode, the focal spots cooperating to form the elongate focal area on and extending along the anode.

Example Embodiment 6: The system of any preceding example embodiment, or combinations thereof, wherein the field emission cathode device comprises one or more serially-actuatable field emission cathode elements arranged to each emit electrons toward different serially-contiguous focal spots or different serially-adjacent focal spots on the anode, the serially-contiguous or serially-adjacent focal spots cooperating to form the elongate focal area on and extending along the anode.

Example Embodiment 7: The system of any preceding example embodiment, or combinations thereof, wherein the field emission cathode device comprises one or more selectively-actuatable field emission cathode elements arranged to emit electrons toward two or more serially-contiguous focal spots or two or more serially-adjacent focal spots on the anode, the two or more serially-contiguous or the two or more serially-adjacent focal spots forming a group of focal spots, with different groups of focal spots cooperating to form the elongate focal area on and extending along the anode.

Example Embodiment 8: The system of any preceding example embodiment, or combinations thereof, wherein the different groups of focal spots are serially-contiguous, are serially-adjacent, or serially-overlap to form the elongate focal area on and extending along the anode.

Example Embodiment 9: The system of any preceding example embodiment, or combinations thereof, wherein the collimator device is configured to shape the X-ray beams by allowing therethrough X-ray beams emitted from the elongate focal area of the anode and directed toward the treatment field.

Example Embodiment 10: The system of any preceding example embodiment, or combinations thereof, wherein the collimator device is configured to shape the X-ray beams by preventing from passing therethrough X-ray beams emitted from the elongate focal area of the anode and not directed toward the treatment field.

Example Embodiment 11: The system of any preceding example embodiment, or combinations thereof, wherein the elongate focal area on and extending along the anode comprises the serially-contiguous or serially-adjacent focal spots, and wherein the collimator device is configured to shape the X-ray beams by allowing therethrough only the X-ray beams directed toward the treatment field from one or more of the focal spots.

Example Embodiment 12: The system of any preceding example embodiment, or combinations thereof, wherein the elongate focal area on and extending along the anode comprises the serially-contiguous or serially-adjacent focal spots, wherein, over a temporal sequence of actuation events, one or more of the focal spots are actuated to emit X-rays beams in each actuation event, and wherein the collimator device is configured to shape the X-ray beams by allowing therethrough only the X-ray beams directed toward the treatment field in each actuation event.

Example Embodiment 13: The system of any preceding example embodiment, or combinations thereof, wherein the collimator device defines one or more apertures, each aperture corresponding to the one or more of the focal spots actuated to emit X-rays beams in each actuation event, and the one or more apertures being configured to shape the X-ray beams by allowing therethrough only the X-ray beams directed toward the treatment field and emitted from the one or more focal spots in each actuation event, and wherein the collimator device is disposed in a fixed position between the anode and the treatment field, or is movable relative to the anode.

Example Embodiment 14: The system of any preceding example embodiment, or combinations thereof, wherein the collimator device is movable relative to the anode, and the collimator device is a planar member laterally movable relative to the elongate focal area.

Example Embodiment 15: The system of any preceding example embodiment, or combinations thereof, wherein the collimator device is movable relative to the anode, and the collimator device is an arcuate member movable relative to the elongate focal area in an orbit around the treatment field.

Example Embodiment 16: The system of any preceding example embodiment, or combinations thereof, wherein the collimator device is movable relative to the anode, and the collimator device is an annular member movable relative to the elongate focal area via rotation around the treatment field.

Example Embodiment 17: The system of any preceding example embodiment, or combinations thereof, wherein the collimator device defines one or more apertures, each aperture corresponding to the one or more of the focal spots actuated to emit X-rays beams in each actuation event, and wherein the one or more apertures of the collimator device are configured to shape the X-ray beams by allowing therethrough only the X-ray beams directed toward the treatment field and emitted from the one or more focal spots in each actuation event.

Example Embodiment 18: The system of any preceding example embodiment, or combinations thereof, further comprising an X-ray detector arranged to oppose the collimator device such that the treatment field is disposed between the collimator device and the X-ray detector, wherein the X-ray detector is further arranged to receive the X-ray beams directed through the collimator device and interacting with the treatment field.

Example Embodiment 19: The system of any preceding example embodiment, or combinations thereof, further comprising a controller in communication with the X-ray detector, and configured to form an X-ray image of the treatment field from the X-ray beams received by the X-ray detector.

Example Embodiment 20: A method for providing an X-ray dosage, the method comprising forming an elongate focal area on and extending along an anode by emitting, using an X-ray source including the anode and a field emission cathode device in spaced-apart relation with the anode, electrons from the field emission cathode device toward the anode, the emitted electrons interacting with the anode to form the elongate focal area and actuating emission of X-ray beams from the elongate focal area toward a treatment field; and shaping, using a collimator device disposed between the anode and the treatment field, the X-ray beams from the elongate focal area of the anode by filtering the X-ray beams to minimize a penumbra of the X-ray beams directed through the collimator device to interact with the treatment field.

Example Embodiment 21: The method of any preceding example embodiment, or combinations thereof, wherein forming the elongate focal area comprises forming the elongate focal area on and extending along the anode such that the elongate focal area defines a major dimension and a minor dimension, with the major dimension being greater than the minor dimension.

Example Embodiment 22: The method of any preceding example embodiment, or combinations thereof, wherein the field emission cathode device comprises one or more selectively-actuatable field emission cathode elements arranged to emit electrons toward the anode, and wherein forming the elongate focal area comprises selectively actuating one or more of the field emission cathode elements to form the elongate focal area on and extending along the anode.

Example Embodiment 23: The method of any preceding example embodiment, or combinations thereof, wherein the field emission cathode device comprises one or more selectively-actuatable field emission cathode elements arranged to each emit electrons toward the anode, and wherein forming the elongate focal area comprises selectively actuating one or more of the field emission cathode elements to direct the electrons emitted therefrom toward different serially-contiguous focal spots or different serially-adjacent focal spots on the anode, the serially-contiguous or serially-adjacent focal spots forming the elongate focal area on and extending along the anode.

Example Embodiment 24: The method of any preceding example embodiment, or combinations thereof, wherein the field emission cathode device comprises one or more selectively-actuatable field emission cathode elements arranged to emit electrons toward the anode, and wherein forming the elongate focal area comprises selectively actuating one or more of the field emission cathode elements to direct the electrons emitted therefrom toward different focal spots on the anode, the focal spots cooperating to form the elongate focal area on and extending along the anode.

Example Embodiment 25: The method of any preceding example embodiment, or combinations thereof, wherein the field emission cathode device comprises one or more serially-actuatable field emission cathode elements arranged to each emit electrons toward the anode, and wherein forming the elongate focal area comprises selectively actuating one or more of the field emission cathode elements to direct the electrons emitted therefrom toward different serially-contiguous focal spots or different serially-adjacent focal spots on the anode, the serially-contiguous or serially-adjacent focal spots cooperating to form the elongate focal area on and extending along the anode.

Example Embodiment 26: The method of any preceding example embodiment, or combinations thereof, wherein the field emission cathode device comprises one or more selectively-actuatable field emission cathode elements arranged to emit electrons toward the anode, and wherein forming the elongate focal area comprises selectively actuating one or more of the field emission cathode elements to direct the electrons emitted therefrom toward two or more serially-contiguous focal spots or two or more serially-adjacent focal spots on the anode, the two or more serially-contiguous or the two or more serially-adjacent focal spots forming a group of focal spots, with different groups of focal spots cooperating to form the elongate focal area on and extending along the anode.

Example Embodiment 27: The method of any preceding example embodiment, or combinations thereof, comprising forming the elongate focal area from the different groups of focal spots, wherein the different groups of focal spots are selected from the group consisting of serially-contiguous groups of focal spots, serially-adjacent groups of focal spots, serially-overlapping groups of focal spots, and combinations thereof.

Example Embodiment 28: The method of any preceding example embodiment, or combinations thereof, wherein shaping the X-ray beams comprises shaping the X-ray beams using the collimator device configured to allow therethrough X-ray beams emitted from the elongate focal area of the anode and directed toward the treatment field.

Example Embodiment 29: The method of any preceding example embodiment, or combinations thereof, wherein shaping the X-ray beams comprises shaping the X-ray beams using the collimator device configured to prevent from passing therethrough X-ray beams emitted from the elongate focal area of the anode and not directed toward the treatment field.

Example Embodiment 30: The method of any preceding example embodiment, or combinations thereof, wherein the elongate focal area on and extending along the anode comprises the serially-contiguous or serially-adjacent focal spots, and wherein shaping the X-ray beams comprises shaping the X-ray beams using the collimator device configured to allow therethrough only the X-ray beams directed toward the treatment field from one or more of the focal spots.

Example Embodiment 31: The method of any preceding example embodiment, or combinations thereof, wherein the elongate focal area on and extending along the anode comprises the serially-contiguous or serially-adjacent focal spots, wherein, over a temporal sequence of actuation events, one or more of the focal spots are actuated to emit X-rays beams in each actuation event, and wherein shaping the X-ray beams comprises shaping the X-ray beams using the collimator device configured to allow therethrough only the X-ray beams directed toward the treatment field in each actuation event.

Example Embodiment 32: The method of any preceding example embodiment, or combinations thereof, wherein the collimator device defines one or more apertures, each aperture corresponding to the one or more of the focal spots actuated to emit X-rays beams in each actuation event, and the one or more apertures being configured to allow therethrough only the X-ray beams directed toward the treatment field and emitted from the one or more focal spots in each actuation event, and wherein shaping the X-ray beams comprises shaping the X-ray beams using the collimator device disposed in a fixed position between the anode and the treatment field, or movable relative to the anode.

Example Embodiment 33: The method of any preceding example embodiment, or combinations thereof, wherein the collimator device is movable relative to the anode, and wherein shaping the X-ray beams comprises shaping the X-ray beams using the collimator device configured as a planar member laterally movable relative to the elongate focal area.

Example Embodiment 34: The method of any preceding example embodiment, or combinations thereof, wherein the collimator device is movable relative to the anode, and wherein shaping the X-ray beams comprises shaping the X-ray beams using the collimator device configured as an arcuate member movable relative to the elongate focal area in an orbit around the treatment field.

Example Embodiment 35: The method of any preceding example embodiment, or combinations thereof, wherein the collimator device is movable relative to the anode, and wherein shaping the X-ray beams comprises shaping the X-ray beams using the collimator device configured as an annular member movable relative to the elongate focal area via rotation around the treatment field.

Example Embodiment 36: The method of any preceding example embodiment, or combinations thereof, wherein the collimator device defines one or more apertures, each aperture corresponding to the one or more of the focal spots actuated to emit X-rays beams in each actuation event, and wherein shaping the X-ray beams comprises shaping the X-ray beams using the collimator device having the one or more apertures of the collimator device configured to allow therethrough only the X-ray beams directed toward the treatment field and emitted from the one or more focal spots in each actuation event.

Example Embodiment 37: The method of any preceding example embodiment, or combinations thereof, further comprising receiving, using an X-ray detector arranged to oppose the collimator device such that the treatment field is disposed between the collimator device and the X-ray detector, the X-ray beams directed through the collimator device and interacting with the treatment field.

Example Embodiment 38: The method of any preceding example embodiment, or combinations thereof, further comprising forming, using a controller in communication with the X-ray detector, an X-ray image of the treatment field from the X-ray beams received by the X-ray detector.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure, regardless of whether such features or elements are expressly combined or otherwise recited in a specific embodiment description herein. This disclosure is intended to be read holistically such that any separable features or elements of the disclosure, in any of its aspects and embodiments, should be viewed as intended, namely to be combinable, unless the context of the disclosure clearly dictates otherwise.

Many X-ray applications, such as radiographic imaging and computed tomography, utilize and require an X-ray source having relatively small focal spot(s) (e.g., dimensions on the order of millimeters). While the actuated focal spot emits photons in all directions, the X-rays output from the focal spot are typically manipulated (e.g., shaped) to illuminate only the desired field of view and to minimize dosage to the area surrounding the treatment field. With relatively small focal spots, a metal structure can be formed which collimates the beam with relatively high efficiency and reduced/minimal penumbra. However, the relatively small focal spot area on the anode limits the total power of the electron beam that can be applied before the electron beam melts or otherwise thermally degrades the material of the target/anode. In order to operate the X-ray source at a higher power, one possible solution is to implement a rotating or otherwise moving target/anode, thus spreading the power (and thus the heat load on the anode) over larger area of the anode. Alternatively, the focal spot area can be extended in a linear fashion to reduce the overall power density at the focal spot. Such a relatively large or larger focal spot, sometimes referred to as a focal line or Line Focus X-ray source (LFX), is capable of producing the high flux (dose/dosage rate) at standard 100 SAD (source to axis distance) or other defined distance required for radiotherapy applications. One issue is that the X-rays emitted by such an anisotropic source (LFX) are difficult to collimate, particularly in regard to the long dimension along the focal line, without reducing the overall efficiency of the X-rays interacting with the FOV/treatment field. Aspects of the present disclosure thus provide conformal collimation along the major dimension of the focal line of a LFX, while also increasing or maximizing the X-ray fluence delivered to the target field of view (FOV)/treatment field. Aspects of the present disclosure thus provide a solution for collimation of X-rays from an X-ray source having a relatively large focal spot dimension (e.g., on the order of centimeters).

It will be appreciated that the summary herein is provided merely for purposes of summarizing some example aspects so as to provide a basic understanding of the disclosure. As such, it will be appreciated that the above described example aspects are merely examples and should not be construed to narrow the scope or spirit of the disclosure in any way. It will be appreciated that the scope of the disclosure encompasses many potential aspects, some of which will be further described below, in addition to those herein summarized. Further, other aspects and advantages of such aspects disclosed herein will become apparent from the following detailed description taken in conjunction with the accompanying drawings which illustrate, by way of example, the principles of the described aspects.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1A schematically illustrates an example prior art technique for X-ray tube anode thermal management using passive heat transfer through thermal conduction associated with the material forming the anode;

FIG. 1B schematically illustrates an example prior art technique for X-ray tube anode thermal management using movement (e.g., rotation) of the anode to increase the area of the anode heated by bombardment with the electron beam;

FIG. 1C schematically illustrates an example prior art technique for X-ray tube anode thermal management using active removal of heat from the anode via a liquid circulated through a cooling channel within the anode;

FIGS. 2A and 2B schematically illustrate prior art X-ray sources, wherein a Line Focus X-ray source (LFX) (FIG. 2B) can be implemented to increase the total radiation output over traditional X-ray sources (FIG. 2A) by maintaining a high electron power density over a relatively larger area on the anode;

FIGS. 3A and 3B schematically illustrate prior art X-ray sources, and the X-ray beam geometry and penumbra resulting from a differing focal spot dimension, wherein a relatively small focal spot minimizes the penumbra (FIG. 3A), and wherein a Line Focus source with relatively larger area of the focal spot can generate a higher X-ray output, but also result in a wide beam penumbra region (FIG. 3B);

FIGS. 4A and 4B schematically illustrate an X-ray source according to one aspect of the present disclosure, wherein the field emission cathode device comprises a plurality of individually controllable field emission cathodes, wherein one of the field emission cathodes can be actuated to provide an electron beam to one focal spot on the anode (FIG. 2A), and then that field emission cathode de-actuated and another one of the field emission cathodes actuated to provide an electron beam to a different focal spot on the anode (FIG. 2B);

FIG. 5 schematically illustrates an X-ray source according to the aspect of the present disclosure shown in FIGS. 4A and 4B, wherein individual field emission cathodes (or small groups of field emission cathodes) can be sequentially actuated to provide electron beams to a different focal spots (or different groups of focal spots) on the anode;

FIG. 6A schematically illustrates an X-ray source according to one aspect of the present disclosure, wherein the field emission cathode device comprises a plurality of individually controllable or actuatable field emission cathodes, wherein many (or all) of the field emission cathodes can be simultaneously actuated to produce a large focal spot/line on the anode for delivery of radiotherapy treatment;

FIGS. 6B and 6C schematically illustrate an X-ray source according to the aspect of the present disclosure shown in FIG. 6A, wherein only some of the field emission cathodes (or some groups of field emission cathodes) are selectively actuated (with the remainder de-actuated) in order to distribute the radiation dose/dosage to the target (FIG. 6B), with the electron current from each field emission cathode or each group of field emission cathodes being modulated to produce intensity modulated radiation (IMRT) from and along the large focal spot/line on the anode;

FIGS. 7A-7D schematically illustrate an X-ray source configured as a Segmented Line Focus X-ray Source (SLFX), according to one aspect of the present disclosure, wherein the SLFX operating principle is illustrated by the sequence from FIG. 7A to FIG. 7D whereby several focal line segments of length L are actuated/de-actuated in a particular sequence to avoid over-temperature of the anode material, while a dynamic collimator moves or is changed in configuration in correspondence with the actuated focal line segment to provide maximal X-ray fluence to the FOV or treatment field, while minimizing the penumbra/penumbra effect;

FIG. 8A-8C schematically illustrate an X-ray source configured as a Segmented Line Focus X-ray Source (SLFX), according to one aspect of the present disclosure, wherein the length of the elongate focal area can be chosen in any increment of two or more serially-contiguous or serially-adjacent focal spots;

FIGS. 9A-9D schematically illustrate an X-ray source configured as a Segmented Line Focus X-ray Source (SLFX), according to one aspect of the present disclosure, wherein the X-ray dosage rate to the FOV/treatment field can be increased by actuating multiple focal line segments (focal spots) simultaneously while, due to the relatively small size of each focal spot, minimizing the penumbra/penumbra effect.

FIGS. 10A and 10B schematically illustrate an X-ray source configured as a Segmented Line Focus X-ray Source (SLFX), according to one aspect of the present disclosure, wherein the SLFX assembly can be associated with a translating collimator (FIG. 10A) or a rotating collimator (FIG. 10B) each being configured to dynamically adjust slot/opening/aperture size and/or position to maintain focus of maximal X-ray fluence (dosage rate) onto the FOV/treatment field;

FIG. 11 schematically illustrates system including three X-ray sources each configured as a Segmented Line Focus X-ray Source (SLFX), according to one aspect of the present disclosure, wherein the three SLFX assemblies each with a dedicated collimator can be disposed to surrounding the FOV/treatment field, and to have multiple X-ray beams simultaneously actuated to provide radiation treatment to the FOV/treatment field;

FIGS. 12A and 12B schematically illustrate an X-ray source configured as a Segmented Line Focus X-ray Source (SLFX), according to one aspect of the present disclosure, wherein the SLFX assembly is associated with a rotating annular collimator and a planar X-ray detector (FIG. 12A) or an arcuate X-ray detector (FIG. 12B) disposed within the annular collimator so as to provide imaging capabilities for the FOV/treatment field; and

FIG. 13 schematically illustrates a method for providing an X-ray dosage, according to one aspect of the present disclosure.

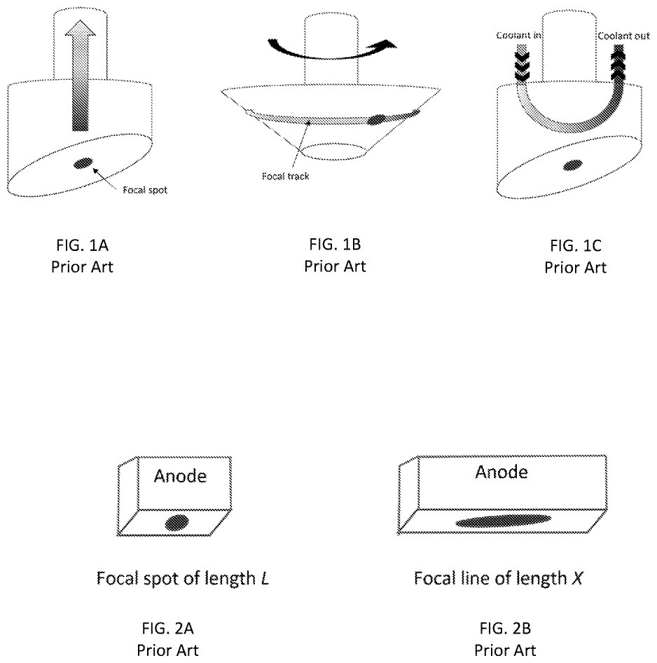

### DETAILED DESCRIPTION OF THE DISCLOSURE

The present disclosure now will be described more fully hereinafter with reference to the accompanying drawings, in which some, but not all embodiments of the disclosures are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

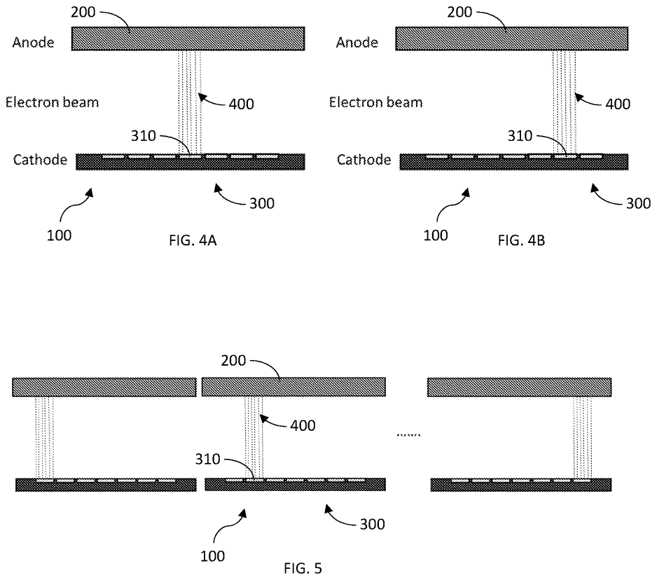

One aspect of the present disclosure comprises a system for providing an X-ray dosage (e.g., otherwise referred to herein as an X-ray system). In some aspects, as shown for example in FIGS. 4A and 4B, such a system comprises an X-ray source 100 including an anode 200 and a field emission cathode device 300 in spaced-apart relation with the anode 200 and arranged to emit electrons (e.g., an electron beam 305) toward the anode 200 so as to form an elongate focal area 400 on and extending along the anode 200.

In one aspect, the field emission cathode device 300 comprises a plurality of individually-controllable field emission cathodes 310. As such, since an X-ray imaging application generally requires a small focal spot size on the anode 200 to provide higher resolution for the imaging, the X-ray source 100 can be operated in an imaging mode, wherein only one field emission cathode 310 or a few, several, or otherwise a small number of field emission cathodes 310 of the field emission cathode device 300 can be actuated to achieve a relatively small focal spot (e.g., sub-millimeter to a few millimeters in diameter) of the electron beam(s) 305 on the anode 200 for generating X-ray beams for imaging purposes (see, e.g., FIG. 4A). In some configurations of the X-ray source 100, a different single field emission cathode 310 (or a different few, several, or otherwise a small number of field emission cathodes 310) may be actuated to achieve another relatively small focal spot (e.g., sub-millimeter to a few millimeters in diameter) of the electron beam(s) 305 on another portion of the anode 200, as shown in FIG. 4B. In the imaging mode, the energy of the X-ray source (e.g., the energy is proportional to the voltage applied to the anode 200) can be adjusted to an appropriate range for imaging (e.g., about 30 kV-160 kV) to achieve suitable X-ray imaging results. In some aspects, as shown for example in FIG. 5, multiple views of the target can be obtained from various viewing angles, for example, for three-dimensional (3D) imaging purposes. That is, as shown in FIG. 5, groups of one or a few field emission cathodes can be sequentially actuated to produce a series of X-ray images from different viewing angles (e.g., the electron beam(s) from the field emission cathodes interact with different portions/focal spots of the anode, wherein each focal spot directs the X-ray beam(s) toward the target object at different angles). Those images can be processed and combined, as appropriate, to reconstructed and achieve a 3D view of the target object within the field of view/treatment field.

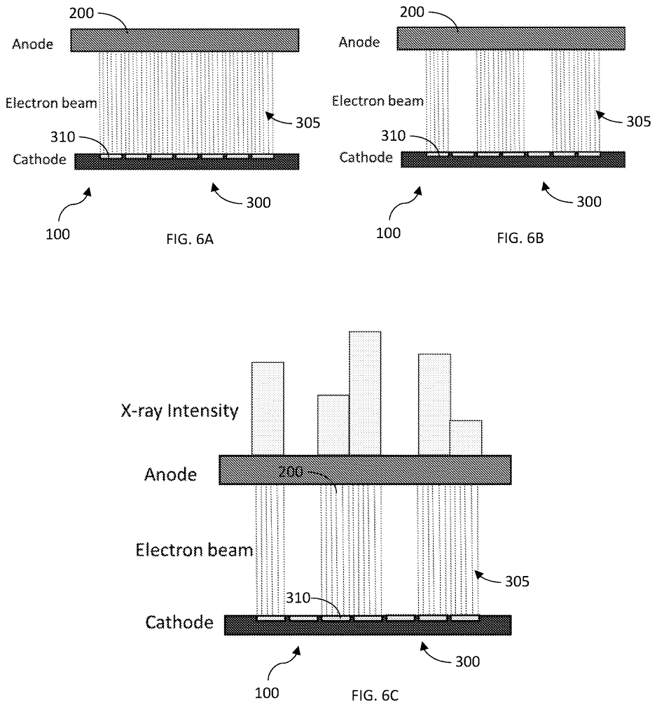

In another aspect of the present disclosure, the X-ray source 100 can be operated in a treatment mode or power mode, in order to produce a relatively large focal spot of the electron beam(s) 305 on the anode 200. That is, in some instances, a large number (or all) of the plurality of field emission cathodes 310 can be actuated simultaneously (or selectively or serially), with the respective emitted electron beams being directed toward adjacent or overlapping focal spots on the anode 200, such that focal spots/areas of the anode 200 impacted by the electron beams 305 combine to form a joint large focal spot or focal line (e.g., up to the order of 10's of centimeters) on the anode 200, as shown for example in FIG. 6A. The large focal spot/line allows the X-ray source 100 to be operated at relatively higher power to provide a proportionally higher radiation dose/dosage rate (e.g., over 40 Gy/sec at standard 100 cm source to axis distance (100 SAD)) as an output for a FOV/treatment field. For example, the energy of the X-ray source 100 is increased by increasing the voltage applied to the anode 200 to a relatively higher level (e.g., about 160 kV-800 kV) to achieve a suitable radiation dose/dosage rate distribution across the target object for better treatment outcomes.

In some aspects, the X-ray source 100 in the treatment/power mode may have only some of the field emission cathodes 310 selectively actuated, while the remainder of the field emission cathodes 310 remain de-actuated, as shown for example in FIG. 6B. In addition, the current (electron flow in the electron beam) from each of the actuated field emission cathodes (and thus the X-ray radiation emitted from the corresponding focal spot on the anode 200) can be programmed/modulated to have different intensity levels, as shown for example in FIG. 6C. This selective field emission cathode actuation and electron beam modulation operational mode of the X-ray source 100 results in different radiation intensities from the corresponding focal spots forming the joint large focal spot or focal line, as shown in FIG. 6C.

Aspects of such an X-ray source 100 may involve, for example, the field emission cathode device 300 being switchable between an imaging mode having the emitted electrons/electron beam 305 focused on a first focal area on the anode 200, wherein the first focal area has a first focal area size, and a power mode or treatment mode having the emitted electrons/electron beam 305 focused on a second focal area on the anode 200, wherein the second focal area has a second focal area size greater than the first focal area size.

In one aspect, the field emission cathode device 300 comprises a plurality of individually-controllable field emission cathodes 310 configured and arranged such that the imaging mode comprises a first amount of the field emission cathodes 310 that are actuated to direct the electrons emitted thereby (electron beam 305) to the first focal area on the anode 200. In a further aspect, the field emission cathode device 300 is configured such that the power mode comprises a second amount of the field emission cathodes 310 that are actuated to direct the electrons emitted thereby (electron beam 310) to the second focal area on the anode 200. The second amount of the field emission cathodes 310 is greater than the first amount of the field emission cathodes 310. Further, the second amount of field emission cathodes 310 is arranged to direct the electrons emitted thereby (electron beam or current) to respective corresponding focal areas on the anode 200, wherein the respective corresponding focal areas are adjacently disposed on the anode 200, and are arranged to form the second focal area on the anode 200. In further aspects, each cathode of the second amount of the field emission cathodes 310 is actuated to provide a current of electrons/an electron beam directed to the anode 200, and wherein the current from a selected one or more of the second amount of field emission cathodes 310 is modulatable so as to modulate an intensity of the electrons emitted thereby to the second focal area. As such, the first and second amounts of the field emission cathodes 305 in the field emission cathode device 300 can be selected and arranged to provide the appropriate focal spot size on the anode 200 for the imaging and treatment/power modes, respectively, with the appropriate energy for each mode being determined based on the voltage applied to the anode 200.

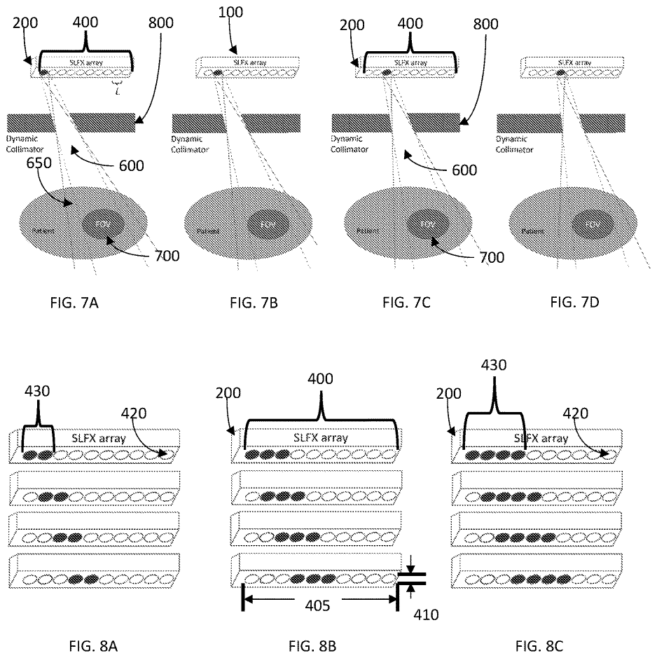

Given the disclosure herein that various aspects of the present disclosure implement a field emission cathode device 300 comprising a plurality of individually-controllable field emission cathodes 310 configured and arranged to be selectively actuated such that the electrons emitted thereby (electron beam(s) 305) are directed to a corresponding focal spot/area on the anode 200, the field emission cathode device 300 is not included in FIGS. 7A-7D, 8A-8C, 9A-9D, 10A-10B, 11, and 12A-12B for clarity purposes. A person of skill in the art will thus understand and appreciate that the descriptions of those noted figures are expressly premised upon the individually-controllable field emission cathodes 310 being configured and arranged to be selectively actuated such that the electrons emitted thereby (electron beam(s) 305) are directed to a corresponding focal spot/area on the anode 200 so as to actuate the focal spot(s) on the anode 200, as discussed further herein. As such, actuation of the focal spot(s) in connection with actuation of the elongate focal area 400, expressly refers to the X-ray beam(s) generated by the interaction of the electron beam(s) 305 from the field emission cathodes 310, with the corresponding focal spot(s)/area(s) of the anode 200, which actuate the corresponding focal spot(s)/area(s) forming the elongate focal area 400.

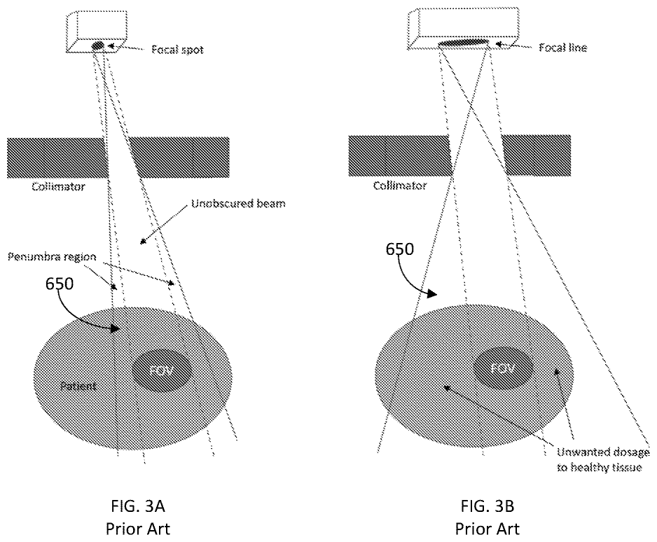

Accordingly, as shown for example in FIGS. 7A-7D, aspects of the present disclosure demonstrate an interaction between the emitted electrons/the electron beam(s) 305 and the anode 200 that forms/defines the elongate focal area 400 (see, e.g., FIG. 7A) on the anode 200, and actuates emission of X-ray beams 600 from the elongate focal area 400. The resulting X-ray beams 600 are directed toward a treatment field 700 external to (e.g., separate, discrete, and/or spaced apart from) the X-ray source 100. A collimator device 800 (otherwise referred to herein as a collimator 800) is disposed between the anode 200 and the treatment field 700 (wherein the collimator device 800 can be a component of the system separate from the X-ray source 100 or can otherwise be associated with and/or be a component of the X-ray source 100). In some aspects, the collimator device 800 is configured to shape the X-ray beams 600 emitted from the elongate focal area 400 of the anode 200 by filtering the X-ray beams 600 to reduce or minimize a penumbra 650 (e.g., as illustrated, for example, in FIGS. 3A, 3B, and 7A) of the X-ray beams 600 directed through the collimator device 800 to interact with the treatment field 700.

In some aspects, implementing a line X-ray source or a discrete X-ray source with high packing density (small pitch) of focal spots may render it difficult or impractical to also implement a static collimator that can accommodate/shape the X-ray beams from all focal spots due to spatial limitations of such a static collimator (e.g., given the physical constraints of forming a finite number of openings/apertures in a limited area. Accordingly, some aspects of the present disclosure herein can implement a dynamic collimator to reduce or eliminate limitations of a static collimator, though a static collimator or a dynamic/static hybrid collimator can otherwise be implemented as necessary or desired, as further disclosed herein. In particular aspects, the elongate focal area 400 on and extending along the anode 200 defines a major dimension 405 and a minor dimension 410 (see, e.g., FIG. 8B), with the major dimension 405 being greater than the minor dimension 410. In some aspects, the elongate focal area 400 can define, for example, an oval-shaped focal area, a stadium or pill-shaped focal area, a linear focal area, or an approximately linear focal area. In addition, the field emission cathode device 300 comprises one or more individually-controllable field emission cathodes 310 (otherwise referred to herein as field emission cathode elements 310) that are selectively-actuatable, and arranged to emit the electrons 305 toward the anode 200 to form the elongate focal area 400 on and extending along the anode 200 such that the electrons 305 actuate one or more of the focal spots of the elongate focal area 400.

More particularly, in such aspects as shown in FIGS. 7A-7D, an array of individually actuatable focal spots 420, or focal spot segments 430 comprising two or more focal spots 420, are distributed along the length of the elongate focal area 400. The elongate focal area 400 (otherwise referred to herein as a focal line) comprises N focal spots 420 or M segments 430 of two or more focal spots 420, each having a length L and being arranged in a row. Each focal spot 420 or segment 430 can be actuated/de-actuated as controlled by appropriate software-controlled timing circuitry (i.e., to form a Segmented Line Focus X-ray Source (SLFX)). By actuating/de-actuating the focal spots 420 or segments 430 in a particular sequence (i.e., rather than or instead of simultaneously), the actuated portion/area of the elongate focal area 400 shifts/moves along the surface of the anode 200. By frequently changing the actuated area (actuated elongate focal area segment) of the anode 200, effective thermal management is achieved by avoiding overheating of any particular spot on the anode surface while avoiding movement of the anode during operation. In some instances, higher power (e.g., an increased electron beam current directed to the anode 200) can be implemented over the entire length of the elongate focal area 400. When coordinated/implemented with a matching dynamic collimator 800 (e.g., a collimator that moves or is changed in configuration in correspondence with the actuated focal line segment to shape the X-ray beams to provide maximal X-ray fluence to the FOV or treatment field, while minimizing the penumbra/penumbra effect), a high X-ray flux or dosage rate capability is attainable at standard 100 SAD (100 cm source to axis distance) or other defined distance, while preserving the ability to collimate or shape the X-ray beam(s) 600.

In some aspects, the field emission cathode device 300 comprises one or more selectively-actuatable (in some aspects, serially-actuatable) field emission cathode elements 310 arranged to each emit electrons 305 toward different serially-contiguous focal spots 420 or different serially-adjacent focal spots 420 on the anode 200 (e.g., the individual focal spots 420 can be disposed serially with the perimeters thereof in contact with each other, or the individual focal spots 420 can be disposed serially with a space between adjacent perimeters thereof), with the serially-contiguous or serially-adjacent focal spots 420 forming the elongate focal area 400 on and extending along the anode 200.

In other aspects, the field emission cathode device 300 comprises one or more selectively-actuatable field emission cathode elements 310 arranged to emit electrons 305 toward two or more serially-contiguous focal spots 420 or two or more serially-adjacent focal spots 420 on the anode 200, with the two or more serially-contiguous or the two or more serially-adjacent focal spots 420 forming a group or segment of focal spots 430, and with different groups of focal spots 430 cooperating to form the elongate focal area 400 on and extending along the anode 200. In some instances, the different groups of focal spots 430 are serially-contiguous, are serially-adjacent, or serially-overlap to form the elongate focal area 400 on and extending along the anode 200.

That is, in some instances, the length of the elongate focal area 400 can be selected in increments of L, wherein L comprises one or more focal spots 420, as shown for example in each of FIGS. 8A-8C. In instances where L>2, the segments 430 of two or more focal spots 420 can be actuated sequentially (e.g., focal spot 1 and focal spot 2 are actuated, followed by actuation of focal spot 3 and focal spot 4 after de-actuation of focal spots 1 and 2), or the actuated segments 430 can overlap by one or more focal spots (e.g., focal spot 1 and focal spot 2 are actuated, followed by actuation of focal spot 2 and focal spot 3 after de-actuation of focal spots 1 and 2). In instances where the FOV or treatment field 700 is relatively large, it may be preferrable to implement a larger focal spot size for the focal spots 420 forming the elongate focal area 400 to achieve a higher X-ray flux/dosage rate at standard 100 SAD (source to axis distance) or other defined distance, even though some increase in the penumbra may occur. Because the collimator 800 is dynamic and adjustable, the length of the elongate focal spot can be selected by appropriate programming, thereby allowing for flexibility in the size of the FOV/treatment field having the radiation therapy applied thereto, while acceptably shaping the X-ray beams 600 to control or minimize the penumbra 650.

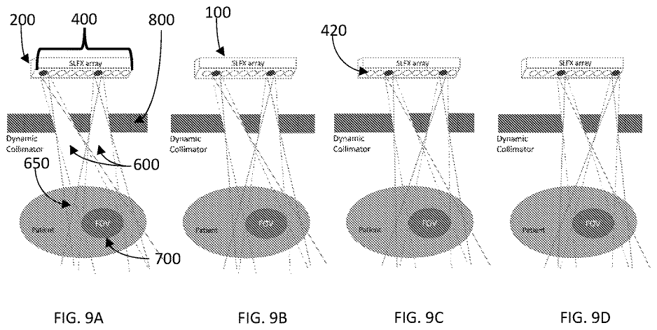

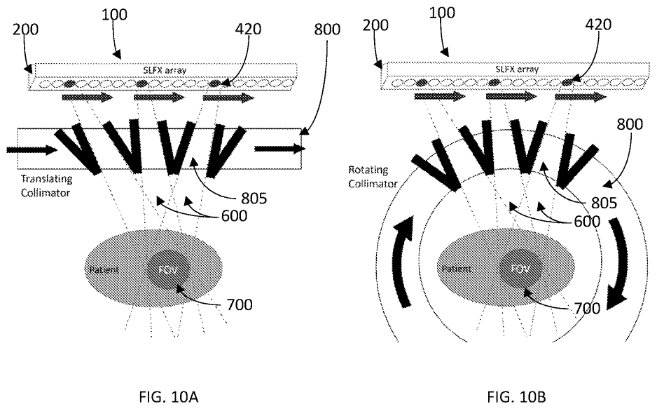

In some aspects, the field emission cathode device 300 comprises one or more selectively-actuatable field emission cathode elements 310 arranged to emit electrons 305 toward different focal spots 420 on the anode 200, with the focal spots 420 cooperating to form the elongate focal area 400 on and extending along the anode 200. That is, since each focal spot 420 or focal spot segment 430 is individually actuatable, more than one focal spot 420 or focal spot segment 430 of the elongate focal area 400 can be actuated at any time, as shown for example in FIGS. 9A-9C. Such an arrangement can serve, for example, to benefit certain radiation therapy applications in that the total X-ray dosage rate at standard 100 SAD (source to axis distance) or other defined distance is increased without a change in the power density, thereby enabling shorter treatment times. In such aspects, a number of different elongate focal areas 400 (e.g., multiple focal lines) can be implemented, as long as each elongate focal area 400 is provided with a corresponding opening/aperture of the conformal dynamic collimator 800. The minimum pitch between the different elongate focal areas 400, in such instances, depends, for example, on the size of the collimator hardware and the extent to which such hardware may interference with X-ray beams from adjacent focal lines.

In some aspects, the collimator device 800 is configured to shape the X-ray beams 600 by allowing therethrough X-ray beams 600 emitted from the elongate focal area 400 of the anode 200 and directed toward the treatment field 700. In some instances, the collimator device 800 is configured to shape the X-ray beams 600 by preventing from passing therethrough X-ray beams 600 emitted from the elongate focal area 400 of the anode 200 and not directed toward the treatment field 700. Essentially, the collimator 800 includes a body defining one or more openings/apertures, wherein the openings/apertures shape the X-ray beams 600 by permitting X-ray beams 600 directed toward the treatment field 700 to pass through the collimator 800 (with minimal penumbra), while the body defining the openings/apertures prevents X-ray beams 600 not directed toward the treatment field 700 from passing through the collimator 800, thus performing a filtering function for the X-ray beams 600.

In some aspects, the collimator 800 is comprised, for example, of metal plates of high-Z X-ray absorbing material such as steel, lead, or tungsten. Because the collimator 800 is generally disposed proximate or in close proximity to the X-ray source 100, a high degree of precision is required in order to maintain accurate positioning of the collimator 800. For a conformal collimator 800 that is disposed in a stationary position relative to the X-ray source 100, sub-millimeter precision of the collimator dimensions can be achieved. However, a moving and/or re-configurable slot/opening/aperture of the collimator 800 may be needed in order to maintain correspondence of the slot/opening/aperture with the shifting actuated focal spot(s) 420 on the surface of the anode 200. The shape of the slot/opening/aperture depends, for example, on the length L of each segment of the elongate focal area 400, and the width or lateral dimension of the desired FOV/treatment field 700. Accordingly, the collimator 800 can be a stationary or movable collimator (relative to the X-ray source 100) defining one or more re-configurable or re-shapeable slot(s)/opening(s)/aperture(s) corresponding to the length L of each segment of the elongate focal area 400, and the width or lateral dimension of the desired FOV/treatment field 700.

In some instances, the elongate focal area 400 on and extending along the anode 200 comprises the serially-contiguous or serially-adjacent focal spots 420, wherein the collimator device 800 is configured to shape the X-ray beams 600 by allowing therethrough only the X-ray beams 600 directed toward the treatment field 700 from one or more of the focal spots 420, wherein, over a temporal sequence of actuation events, one or more of the focal spots 420 is/are actuated to emit X-rays beams 600 in each actuation event, and wherein the collimator device 800 is configured to shape the X-ray beams 600 by allowing therethrough only the X-ray beams 600 directed toward the treatment field 700 in each actuation event. In some aspects, the collimator device 800 defines one or more apertures 805, wherein each aperture 805 corresponds to the one or more of the focal spots 420 actuated to emit X-rays beams 600 in each actuation event, wherein the one or more apertures 805 is configured to shape the X-ray beams 600 by allowing therethrough only the X-ray beams 600 directed toward the treatment field 700 and emitted from the one or more focal spots 420 in each actuation event. The collimator device 800 can be disposed in a fixed position between the anode 200 and the treatment field 700, or can be movable relative to the anode 200.

In one aspect, the re-configurable or re-shapeable slot(s)/opening(s)/aperture(s) 805 of the collimator 800, for and corresponding to multiple segments of the elongate focal area 400, can be associated with a collimator 800 that can be laterally shifted or caused to translate position (in any direction), as needed, in real-time during the treatment procedure (see, e.g., FIG. 10A). That is, in some instances, the collimator device 800 is movable relative to the anode 200, and the collimator device 800 is a planar member laterally movable relative to the elongate focal area 400.

In another aspect, the collimator device 800 is movable relative to the anode 200, and the collimator device 800 is an arcuate member movable relative to the elongate focal area 400 in an orbit around the treatment field 700, or the collimator device 800 is an annular member movable relative to the elongate focal area 400 via rotation around the treatment field 700. In this manner, the re-configurable or re-shapeable slot(s)/opening(s)/aperture(s) 805 of the collimator 800 orbit or rotate about the FOV/treatment field 700 in relation to the orbiting/rotating axis of the collimator 800 (see, e.g., FIG. 10B).

Movement of the collimator 800 can be cyclical, reciprocating, or continuously rotating, in various aspects. In some instances, the collimator 800 can be moved to discrete positions and maintained in each position for actuation(s) of one or more segments of the elongate focal area 400. In this manner, discrete imaging and/or treatment events may be performed. In any event, the actuation sequence of the focal spot segments 430 is coordinated with the position/configuration of the collimator slot(s)/opening(s)/aperture(s) 805 so as to prevent obstruction of the FOV/treatment field 700 during operation.

In one aspect, movement of a movable collimator can be triggered/actuated in association with the control of the actuation of the focal spot(s)/focal spot segment(s). In an alternate aspect, actuation of the focal spot(s)/focal spot segment(s) can be triggered/actuated by optical sensing of the collimator position relative to the X-ray source. Further, in order for the collimator slot(s)/opening(s)/aperture(s) to be re-configurable/re-shapeable, for example, a series of adjacent metal plates or bars movable relative to each other (e.g., as a multi-leaf collimator (MLC)) can be implemented to dynamically shape the radiation flux imparted to the FOV/treatment field, as the actuated segment of the elongate focal area changes.

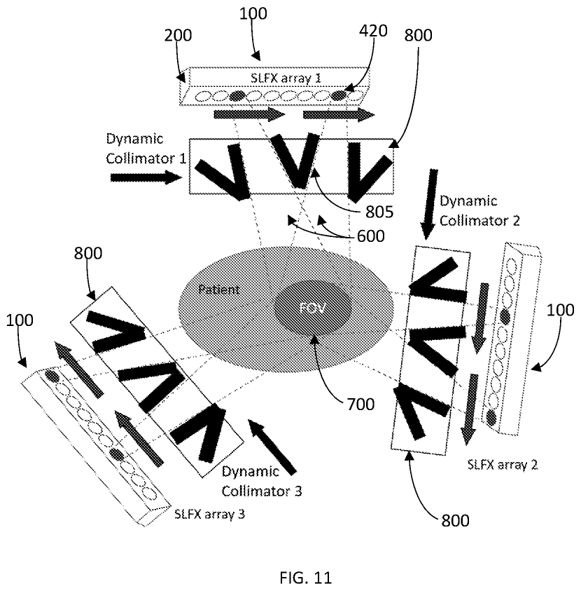

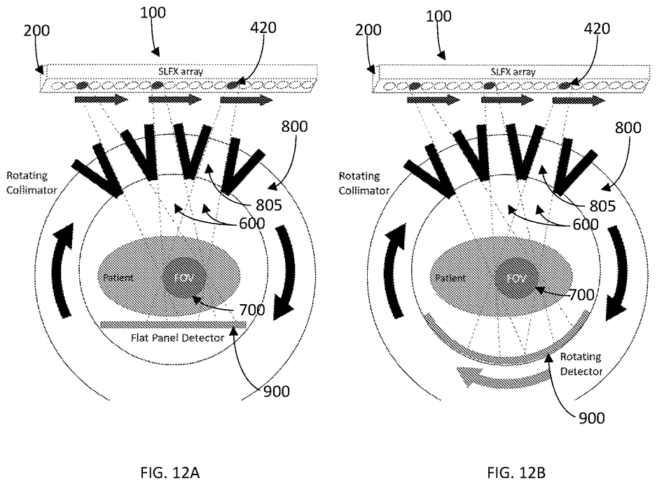

In some aspects, the delivered dosage rate at standard 100 SAD (source to axis distance) or other defined distance can be increased/maximized by implementing multiple systems (X-ray source 100 and conformal collimator 800) disposed in different positions about the FOV/treatment field 700, as shown for example in FIG. 11, such that the X-ray beams 600 are directed to the FOV/treatment field 700 from different directions. In this manner, the X-ray sources 100 can be disposed in stationary positions, and multiple X-ray sources 100 can be implemented to operate simultaneously, thereby reducing treatment time. As shown in FIG. 11, in one example, 3 SLFX assemblies can be disposed about the FOV/treatment field and angularly separated from each other (e.g., by 60 degrees). If each X-ray source 100 is configured to each implement a segment of 2 actuated focal spots, 6 X-ray beams are directed to the FOV/treatment field 700 at any time during course of the treatment. The dynamic conformal collimators 800 can be separate and discrete assemblies for each SLFX assembly, or a single annular collimator can be arranged to rotate around the FOV/treatment field 700 so as to be shared by all 3 X-ray sources 100.

In some aspects, an X-ray detector 900 is arranged to oppose the collimator device 800 such that the treatment field 700 is disposed between the collimator device 800 and the X-ray detector 900, wherein the X-ray detector 900 is further arranged to receive the X-ray beams 600 directed through the collimator device 800 and interacting with the treatment field 700. In some instances, a controller 950 is in communication with the X-ray detector 900, and is configured to form an X-ray image of the treatment field 700 from the X-ray beams 600 received by the X-ray detector 900. That is, in some radiotherapy applications, precise X-ray beam positioning may require some verification of the position of the FOV/treatment field (i.e., tumor).

Accordingly, if the focal line segment length L is sufficiently small, the collimator can be adjusted (e.g., the slot/opening/aperture opened) to permit generation and shaping of a sufficiently broad X-ray beam required for 2D radiography. In such instances, the focal spot/collimator can be held stationary for the imaging procedure, since the power requirements are sufficiently low to prevent melting/thermal degradation of the anode. A flat panel X-ray detector 900 can be placed within the annular collimator on an opposite side of the FOV/treatment site 700 from the X-ray source 100 (see, e.g., FIG. 12A). By using multiple focal spot segments in coordination with the dynamic collimator, multiple views can be acquired if necessary to form a projection dataset for tomosynthesis reconstruction. In yet another aspect, the X-ray detector can be an arcuate element arranged to orbit/rotate about the FOV/treatment site 700 in conjunction with the annular collimator, thereby providing an imaging system similar to that of a CT scanner (see, e.g., FIG. 12B), such that a full CT dataset could be acquired if necessary.

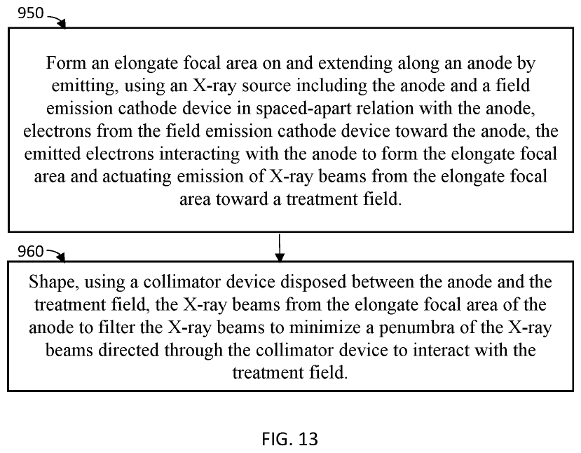

Another aspect of the present disclosure comprises a method for providing an X-ray dosage, as shown for example in FIG. 13. Such a method comprises forming an elongate focal area on and extending along an anode by emitting, using an X-ray source including the anode and a field emission cathode device in spaced-apart relation with the anode, electrons from the field emission cathode device toward the anode, the emitted electrons interacting with the anode to form the elongate focal area and actuating emission of X-ray beams from the elongate focal area toward a treatment field (Block 950); and shaping, using a collimator device disposed between the anode and the treatment field, the X-ray beams from the elongate focal area of the anode to filter the X-ray beams to minimize a penumbra of the X-ray beams directed through the collimator device to interact with the treatment field (Block 960).

Many modifications and other embodiments of the disclosures set forth herein will come to mind to one skilled in the art to which these disclosures pertain having the benefit of the teachings presented in the foregoing descriptions and the associated drawings. For example, though some aspects disclosed herein implement a static/fixed X-ray source in conjunction with a dynamic/movable collimator, a person of ordinary skill in the art will appreciate that a similar or enhanced effect can also be accomplished and achieved by implementing a translating/rotating (i.e., dynamic) X-ray source or X-ray sources with a static and/or dynamic collimator, by implementing a combination of translating/rotating (i.e., dynamic) collimator(s) and X-ray source(s), or by implementing any suitable combination of a static/dynamic X-ray source and a static/dynamic/static-dynamic hybrid collimator, in accordance with the scope of the disclosure herein. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An X-ray system for providing an X-ray dosage, the X-ray system comprising:
an X-ray source including an anode and a field emission cathode device in spaced-apart relation with the anode and arranged to emit electrons toward the anode so as to form an elongate focal area on and extending along the anode and to actuate emission of X-ray beams from the elongate focal area directed toward a treatment field; and
a collimator device disposed between the anode and the treatment field, the collimator device being configured to shape the X-ray beams from the elongate focal area of the anode by filtering the X-ray beams to minimize a penumbra of the X-ray beams directed through the collimator device to interact with the treatment field.

2. The X-ray system of claim 1, wherein the elongate focal area on and extending along the anode defines a major dimension and a minor dimension, with the major dimension being greater than the minor dimension.

3. The X-ray system of claim 1, wherein the field emission cathode device comprises one or more selectively-actuatable field emission cathode elements arranged to emit electrons toward the anode to form the elongate focal area on and extending along the anode.

4. The X-ray system of claim 1, wherein the field emission cathode device comprises one or more selectively-actuatable field emission cathode elements arranged to each emit electrons toward different serially-contiguous focal spots or different serially-adjacent focal spots on the anode, the serially-contiguous or serially-adjacent focal spots forming the elongate focal area on and extending along the anode.

5. The X-ray system of claim 1, wherein the field emission cathode device comprises one or more selectively-actuatable field emission cathode elements arranged to emit electrons toward different focal spots on the anode, the focal spots cooperating to form the elongate focal area on and extending along the anode.

6. The X-ray system of claim 1, wherein the field emission cathode device comprises one or more serially-actuatable field emission cathode elements arranged to each emit electrons toward different serially-contiguous focal spots or different serially-adjacent focal spots on the anode, the serially-contiguous or serially-adjacent focal spots cooperating to form the elongate focal area on and extending along the anode.

7. The X-ray system of claim 1, wherein the field emission cathode device comprises one or more selectively-actuatable field emission cathode elements arranged to emit electrons toward two or more serially-contiguous focal spots or two or more serially-adjacent focal spots on the anode, the two or more serially-contiguous or the two or more serially-adjacent focal spots forming a group of focal spots, with different groups of focal spots cooperating to form the elongate focal area on and extending along the anode.

8. The X-ray system of claim 7, wherein the different groups of focal spots are serially-contiguous, are serially-adjacent, or serially-overlap to form the elongate focal area on and extending along the anode.

9. The X-ray system of claim 1, wherein the collimator device is configured to shape the X-ray beams by allowing therethrough X-ray beams emitted from the elongate focal area of the anode and directed toward the treatment field.

10. The X-ray system of claim 1, wherein the collimator device is configured to shape the X-ray beams by preventing from passing therethrough X-ray beams emitted from the elongate focal area of the anode and not directed toward the treatment field.

11. The X-ray system of claim 1, wherein the elongate focal area on and extending along the anode comprises the serially-contiguous or serially-adjacent focal spots, and wherein the collimator device is configured to shape the X-ray beams by allowing therethrough only the X-ray beams directed toward the treatment field from one or more of the focal spots.

12. The X-ray system of claim 1, wherein the elongate focal area on and extending along the anode comprises the serially-contiguous or serially-adjacent focal spots, wherein, over a temporal sequence of actuation events, one or more of the focal spots are actuated to emit X-rays beams in each actuation event, and wherein the collimator device is configured to shape the X-ray beams by allowing therethrough only the X-ray beams directed toward the treatment field in each actuation event.

13. The X-ray system of claim 12, wherein the collimator device defines one or more apertures, each aperture corresponding to the one or more of the focal spots actuated to emit X-rays beams in each actuation event, and the one or more apertures being configured to shape the X-ray beams by allowing therethrough only the X-ray beams directed toward the treatment field and emitted from the one or more focal spots in each actuation event, and wherein the collimator device is disposed in a fixed position between the anode and the treatment field, or is movable relative to the anode.

14. The X-ray system of claim 13, wherein the collimator device is movable relative to the anode, and the collimator device is a planar member laterally movable relative to the elongate focal area.

15. The X-ray system of claim 13, wherein the collimator device is movable relative to the anode, and the collimator device is an arcuate member movable relative to the elongate focal area in an orbit around the treatment field.

16. The X-ray system of claim 13, wherein the collimator device is movable relative to the anode, and the collimator device is an annular member movable relative to the elongate focal area via rotation around the treatment field.

17. The X-ray system of claim 12, wherein the collimator device defines one or more apertures, each aperture corresponding to the one or more of the focal spots actuated to emit X-rays beams in each actuation event, and wherein the one or more apertures of the collimator device are configured to shape the X-ray beams by allowing therethrough only the X-ray beams directed toward the treatment field and emitted from the one or more focal spots in each actuation event.

18. The X-ray system of claim 1, further comprising an X-ray detector arranged to oppose the collimator device such that the treatment field is disposed between the collimator device and the X-ray detector, wherein the X-ray detector is further arranged to receive the X-ray beams directed through the collimator device and interacting with the treatment field.

19. The X-ray system of claim 18, further comprising a controller in communication with the X-ray detector, and configured to form an X-ray image of the treatment field from the X-ray beams received by the X-ray detector.

20. A method for providing an X-ray dosage, the method comprising:
forming an elongate focal area on and extending along an anode by emitting, using an X-ray source including the anode and a field emission cathode device in spaced-apart relation with the anode, electrons from the field emission cathode device toward the anode, the emitted electrons interacting with the anode to form the elongate focal area and actuating emission of X-ray beams from the elongate focal area toward a treatment field; and
shaping, using a collimator device disposed between the anode and the treatment field, the X-ray beams from the elongate focal area of the anode by filtering the X-ray beams to minimize a penumbra of the X-ray beams directed through the collimator device to interact with the treatment field.

21. The method of claim 20, wherein forming the elongate focal area comprises forming the elongate focal area on and extending along the anode such that the elongate focal area defines a major dimension and a minor dimension, with the major dimension being greater than the minor dimension.

22. The method of claim 20, wherein the field emission cathode device comprises one or more selectively-actuatable field emission cathode elements arranged to emit electrons toward the anode, and wherein forming the elongate focal area comprises selectively actuating one or more of the field emission cathode elements to form the elongate focal area on and extending along the anode.

23. The method of claim 20, wherein the field emission cathode device comprises one or more selectively-actuatable field emission cathode elements arranged to each emit electrons toward the anode, and wherein forming the elongate focal area comprises selectively actuating one or more of the field emission cathode elements to direct the electrons emitted therefrom toward different serially-contiguous focal spots or different serially-adjacent focal spots on the anode, the serially-contiguous or serially-adjacent focal spots forming the elongate focal area on and extending along the anode.

24. The method of claim 20, wherein the field emission cathode device comprises one or more selectively-actuatable field emission cathode elements arranged to emit electrons toward the anode, and wherein forming the elongate focal area comprises selectively actuating one or more of the field emission cathode elements to direct the electrons emitted therefrom toward different focal spots on the anode, the focal spots cooperating to form the elongate focal area on and extending along the anode.

25. The method of claim 20, wherein the field emission cathode device comprises one or more serially-actuatable field emission cathode elements arranged to each emit electrons toward the anode, and wherein forming the elongate focal area comprises selectively actuating one or more of the field emission cathode elements to direct the electrons emitted therefrom toward different serially-contiguous focal spots or different serially-adjacent focal spots on the anode, the serially-contiguous or serially-adjacent focal spots cooperating to form the elongate focal area on and extending along the anode.

26. The method of claim 20, wherein the field emission cathode device comprises one or more selectively-actuatable field emission cathode elements arranged to emit electrons toward the anode, and wherein forming the elongate focal area comprises selectively actuating one or more of the field emission cathode elements to direct the electrons emitted therefrom toward two or more serially-contiguous focal spots or two or more serially-adjacent focal spots on the anode, the two or more serially-contiguous or the two or more serially-adjacent focal spots forming a group of focal spots, with different groups of focal spots cooperating to form the elongate focal area on and extending along the anode.

27. The method of claim 26, comprising forming the elongate focal area from the different groups of focal spots, wherein the different groups of focal spots are selected from the group consisting of serially-contiguous groups of focal spots, serially-adjacent groups of focal spots, serially-overlapping groups of focal spots, and combinations thereof.

28. The method of claim 20, wherein shaping the X-ray beams comprises shaping the X-ray beams using the collimator device configured to allow therethrough X-ray beams emitted from the elongate focal area of the anode and directed toward the treatment field.

29. The method of claim 20, wherein shaping the X-ray beams comprises shaping the X-ray beams using the collimator device configured to prevent from passing therethrough X-ray beams emitted from the elongate focal area of the anode and not directed toward the treatment field.

30. The method of claim 20, wherein the elongate focal area on and extending along the anode comprises the serially-contiguous or serially-adjacent focal spots, and wherein shaping the X-ray beams comprises shaping the X-ray beams using the collimator device configured to allow therethrough only the X-ray beams directed toward the treatment field from one or more of the focal spots.

31. The method of claim 20, wherein the elongate focal area on and extending along the anode comprises the serially-contiguous or serially-adjacent focal spots, wherein, over a temporal sequence of actuation events, one or more of the focal spots are actuated to emit X-rays beams in each actuation event, and wherein shaping the X-ray beams comprises shaping the X-ray beams using the collimator device configured to allow therethrough only the X-ray beams directed toward the treatment field in each actuation event.

32. The method of claim 31, wherein the collimator device defines one or more apertures, each aperture corresponding to the one or more of the focal spots actuated to emit X-rays beams in each actuation event, and the one or more apertures being configured to allow therethrough only the X-ray beams directed toward the treatment field and emitted from the one or more focal spots in each actuation event, and wherein shaping the X-ray beams comprises shaping the X-ray beams using the collimator device disposed in a fixed position between the anode and the treatment field, or movable relative to the anode.

33. The method of claim 32, wherein the collimator device is movable relative to the anode, and wherein shaping the X-ray beams comprises shaping the X-ray beams using the collimator device configured as a planar member laterally movable relative to the elongate focal area.

34. The method of claim 32, wherein the collimator device is movable relative to the anode, and wherein shaping the X-ray beams comprises shaping the X-ray beams using the collimator device configured as an arcuate member movable relative to the elongate focal area in an orbit around the treatment field.

35. The method of claim 32, wherein the collimator device is movable relative to the anode, and wherein shaping the X-ray beams comprises shaping the X-ray beams using the collimator device configured as an annular member movable relative to the elongate focal area via rotation around the treatment field.

36. The method of claim 31, wherein the collimator device defines one or more apertures, each aperture corresponding to the one or more of the focal spots actuated to emit X-rays beams in each actuation event, and wherein shaping the X-ray beams comprises shaping the X-ray beams using the collimator device having the one or more apertures of the collimator device configured to allow therethrough only the X-ray beams directed toward the treatment field and emitted from the one or more focal spots in each actuation event.

37. The method of claim 20, further comprising receiving, using an X-ray detector arranged to oppose the collimator device such that the treatment field is disposed between the collimator device and the X-ray detector, the X-ray beams directed through the collimator device and interacting with the treatment field.

38. The method of claim 37, further comprising forming, using a controller in communication with the X-ray detector, an X-ray image of the treatment field from the X-ray beams received by the X-ray detector.
